(19) United States
(12) Patent Application Publication
(10) Pub. No.: US 2022/0061418 A1
(43) Pub. Date: Mar. 3, 2022

(54) RESPIRATORY FACE MASK

(71) Applicant: BioSerenity, Paris (FR)

(72) Inventors: Antoine Giovanni, Marseille (FR); Marion Guy, Troyes (FR); Pierre-Yves Frouin, Atlanta, GA (US)

(73) Assignee: BioSerenity, Paris (FR)

(21) Appl. No.: 17/011,717

(22) Filed: Sep. 3, 2020

(51) Int. Cl. A41D 13/11; A61B 1/00; A61B 1/233; A61B 1/24

(52) U.S. Cl. CPC A41D 13/1107; A61B 1/00142; A61B 46/20; A61B 1/24; A61B 1/233

(57) ABSTRACT

A respiratory face mask, or personal protective equipment, having a deformable entry point is described herein. Various examples of the respiratory face mask described herein include a deformable entry point that allows for the insertion and removal of a medical device or other object to provide care to a user wearing the face mask. The respiratory face mask is configured to at least partially maintain a barrier between the interior of the mask and the exterior of the mask, thereby providing some degree of protection for the patient receiving treatment from the environment around them as well as those administering care from germs contained in the exhalation of the patient.

### BACKGROUND

Respiratory face masks, such as surgical masks, N95 respirators, KN95 respirators, a bandana-type face mask, a pull-up face mask, a gaiter mask, cloth masks, surgical mask, European standard FFP2 Mask, and the like are often used to reduce the probability of the introduction of a particulate or biological agent (such as a virus or bacteria) into the mouth and/or nose of the wearer of the mask. Respiratory face masks are a form of personal protective equipment (“PPE”), or medical device in general, worn on the face or head that are intended to cover at least, if not all, of the mouth and nose of the wearer and protect the wearer from particulate, dirt, dust, chemical agents or vapors, viruses, bacteria, and/or germs in general. In some examples, the respiratory face masks may be disposable; however, some respiratory face masks can be cleaned or sterilized for reuse. Likewise, face masks are often used to reduce the probability of the communication of a particulate or biological agent (such as a virus or bacteria) from the mouth and/or nose of the wearer of the mask to another individual or on surfaces in the proximity of the wearer of the mask.

It is with these and other concerns that an improved face mask is described herein.

### BRIEF DESCRIPTION OF THE DRAWINGS

The detailed description is set forth with reference to the accompanying figures. The use of the same reference numbers in different figures indicates similar or identical items or features.

FIG. 1 is an illustration of an example respiratory face mask, in accordance with some examples of the present disclosure.

FIG. 2 is an exploded, frontal view of a respiratory face mask showing an entry location and a deformable entry point, in accordance with some examples of the present disclosure.

FIG. 3 is an illustration of an entry location and deformable entry point formed from foam, in accordance with some examples of the present disclosure.

FIG. 4 is an illustration of an entry location and deformable entry point formed from silicone, or various forms of polymerized polysiloxanes in accordance with some examples of the present disclosure.

FIG. 5 is an illustration of an entry location and deformable entry point formed from a single material, in accordance with some examples of the present disclosure.

FIGS. 6A and 6B are illustrations showing a respiratory face mask that may be used for procedures such as, but not limited to, endoscopy, in accordance with some examples of the present disclosure.

FIGS. 7A and 7B are illustrations showing an alternate design for a respiratory face mask that may be used for procedures such as, but not limited to, endoscopy, in accordance with some examples of the present disclosure.

FIG. 8 is a cross-sectional view of a sealing material that may be used for a removable cover, in accordance with some examples of the present disclosure.

### DETAILED DESCRIPTION

Examples of the present disclosure can comprise a respiratory face mask having a deformable entry point. In some examples, it may be necessary or required to place a respiratory face mask over a person receiving care. For example, prior to the onset of pandemics such as the COVID-19 pandemic of the year of 2020, there was not regularly a significant concern relating to the potential transmission of viruses or bacteria from a person receiving treatment, with the exception of situations in which the doctor was aware of the issue and the patient was quarantined. The concern was typically directed at medical personnel, wherein medical personnel are often required to wear respiratory face masks during care in order to reduce the probability of the transmission of germs such as viruses and bacteria, or other agents such as microscopic entities and dust/dirt. The face of the person receiving treatment is often left exposed.

However, there has been an increasing concern not only for medical personnel to patient transmission of germs, but also for patient to medical personnel transmission of germs. The various aspects of the presently disclosed subject matter provided for a treatment environment in which at least a partial barrier to the transmission of germs is maintained through phases of treatment. In some examples, to provide for the maintenance of a partial barrier during treatment, a respiratory face mask is provided with one or more deformable entry points. The deformable entry points allow for the insertion of medical devices needed for treatment. The types of medical devices that may be used in conjunction with various aspects of the presently disclosed subject matter include, but are not limited to, endoscopes, fibroscopes, thermometers, probes, aspirators, feeding tubes, dental instruments, and the like. It should be noted that the presently disclosed subject matter is not limited to the type of medical device that may be used.

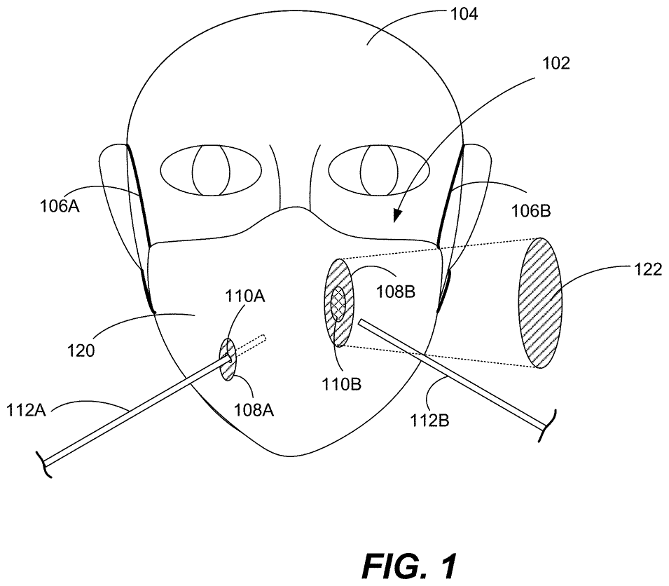

FIG. 1 is an illustration of an example respiratory face mask 102, in accordance with some examples of the present disclosure. The respiratory face mask 102 is designed to be worn over the face of a user 104. The respiratory face mask 102 is removably secured to the face of the user 104 through the use of straps 106A and 106B, which may be place over ears or other securement mechanisms. The presently disclosed subject matter is not limited to any particular way in which the respiratory face mask 102 is secured to the face of the user 104.

To provide for the ability to use medical devices and other equipment while the respiratory face mask 102 is secured to the user 104, the respiratory face mask 102 includes entry locations 108A and 108B. The entry location 108A includes a deformable entry point 110A and the entry location 108B includes a deformable entry point 110B. It should be noted that the inclusion and illustration of multiple entry locations and entry points is merely illustrative, as various aspects of the presently disclosed subject matter may have one entry location or more that two entry locations and one deformable entry point or more than two deformable entry points. Further, it should be understood that the presently disclosed subject matter is not limited to one deformable entry point for one entry location, as various examples of the presently disclosed subject matter may include multiple deformable entry points for one entry location. In some examples, it may be desirable to size the deformable entry point 110A or 110B depending on the particular use. In some examples, for air permeability requirements, the deformable entry points 110A or 110B should be as small as possible. If not, the air permeability may be too low and not in accordance with normative requirements.

The deformable entry points 110A and 110B allow for the insertion and removal of medical devices, such as medical devices 112A and 112B. Medical device 112A is shown inserted through the deformable entry point 110A, while the medical device 112B is shown prior to insertion into or after removal from the deformable entry point 110B. As noted above, the medical devices 112A and 112B may include, but are not limited to, endoscopes, fibroscopes, thermometers, probes, aspirators, feeding tubes, dental instruments, and the like. The presently disclosed subject matter is not limited to any specific type of medical device, or that the device to be inserted be termed a “medical” device, as various aspects of the presently disclosed subject matter may be used for the insertion of various objects. The use of the term “medical device” is not intended to be limiting and is merely being used for the purposes of illustrating various examples of the presently disclosed subject matter.

During use, when inserted through the deformable entry point 110B, the deformable entry point 110B is configured to partially deform around at least a portion of the outside surface of the medical device 112A to provide for at least a partial barrier of air, germs, and the like into and out of the mask 102. While the medical device 112A is being extracted, and after the extraction, from the deformable entry point 110A, the deformable entry point 110A preferable continuously deforms to maintain at least a partial barrier of air, germs, and the like into and out of the mask 102.

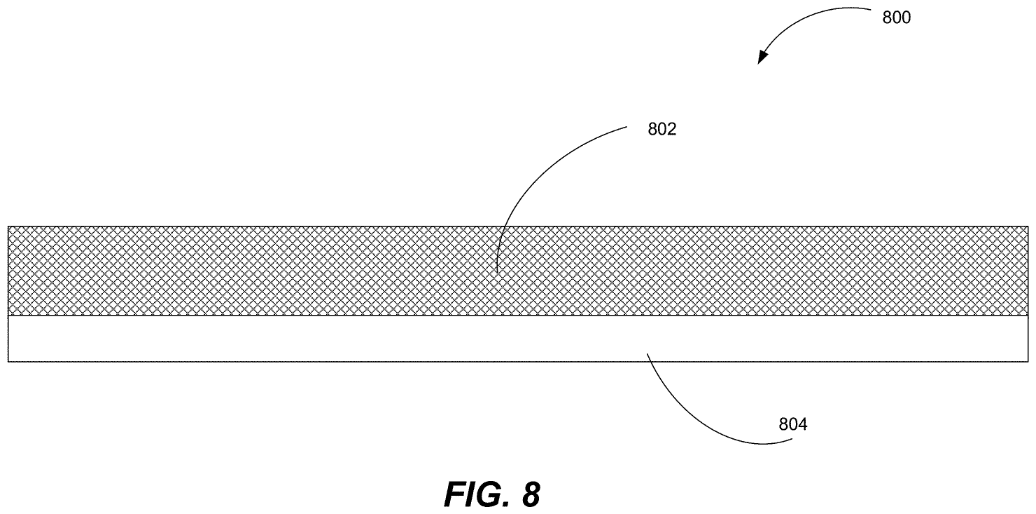

To help, in some examples, maintain the sterility or cleanliness of the area around the deformable entry point 110A, a removable cover 122 may be used. The removable cover 122 may be a removably affixable piece of material, such as cloth, plastic, and the like, that may be placed over at least a portion of the deformable entry point 110A. The removable cover 122 may be affixed using various technologies, including, but not limited to, forms of glue that allow for the removal and replacement of the removable cover 122. In other examples, the removable cover 122 may be formed from a material, such as polyethylene, that uses electrostatic charge (static cling) to affix the removable cover 122. In other examples, the removable cover 122 may be formed from a material, such as hooked strips (e.g. hook and loop fasteners), that uses mechanical cling to affix the removable cover 122. An example of a sealing material used for the removable cover 122 is described in more detail in FIG. 8, below.

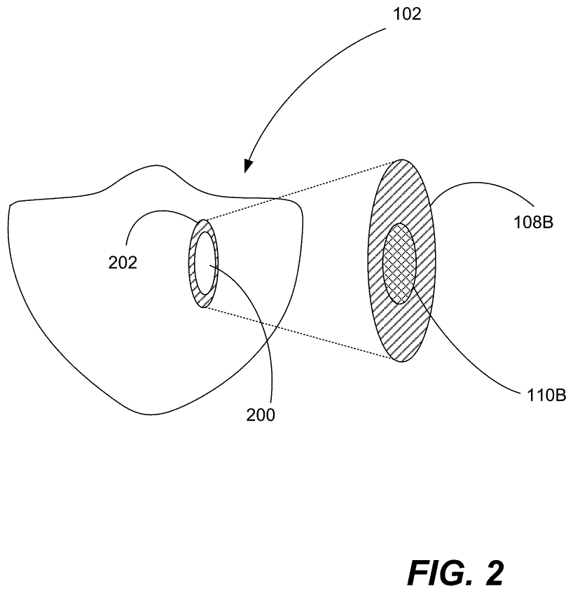

FIG. 2 is an exploded, frontal view of the respiratory face mask 102 of FIG. 1 showing the entry location 108B and the deformable entry point 110B, in accordance with some examples of the present disclosure. In an example of a manner in which the respiratory face mask 102 may be constructed, the respiratory face mask 102 may have stamped out or removed from the material of the respiratory face mask 102 a void 200. The entry location 108B and the deformable entry point 110B may be formed from one piece of material or may be a composite piece of material. The respiratory face mask 102 further includes a sealing location 202. The sealing location 202 is an area around the void 200 that allows the entry location 108B to be affixed to the respiratory face mask 102 if the entry location 108B is a separate piece of material from the respiratory face mask 102. For example, the entry location 108B may be a slit or entry formed into the material of the respiratory face mask 102 itself. If the entry location 108B is to be sealed onto the respiratory face mask 102, the sealing location 202 may include glue or other adhesives. In other examples, the respiratory face mask 102 may be formed from a material that allows the entry location 108B to be heat bonded to the respiratory face mask 102 without the use of glues or adhesives. In some examples, the material comprising the entry location 108B and the deformable entry point 110B may be formed using one material such as foam, plastic, silicone, elastomer, textile and the like.

In some examples, the material comprising the entry location 108B and/or the deformable entry point 110B may be coated with one or more substances to reduce the probability that a germ or other particulate enters or exits the deformable entry point 110B at various times during the use. For example, the material comprising the entry location 108B and/or the deformable entry point 110B may be coated with a gel or other liquid/semi-liquid material that coats at least a portion of the medical device when the medical device is inserted through the deformable entry point 110B. The coating of the gel or other liquid/semi-liquid material onto the medical device may help to maintain at least a partial barrier as the medical device is inserted and removed. In another example, the material comprising the entry location 108B and/or the deformable entry point 110B may be coated with an anti-viral/anti-microbial/anti-bacterial agent that is designed to kill at least a portion of pathogens or germs that may be present at or proximate to the interface of the deformable entry point 110B and the medical device being inserted through the deformable entry point 110B. Various types of anti-viral/anti-microbial/anti-bacterial agents may be used and are considered to be within the scope of the presently disclosed subject matter.

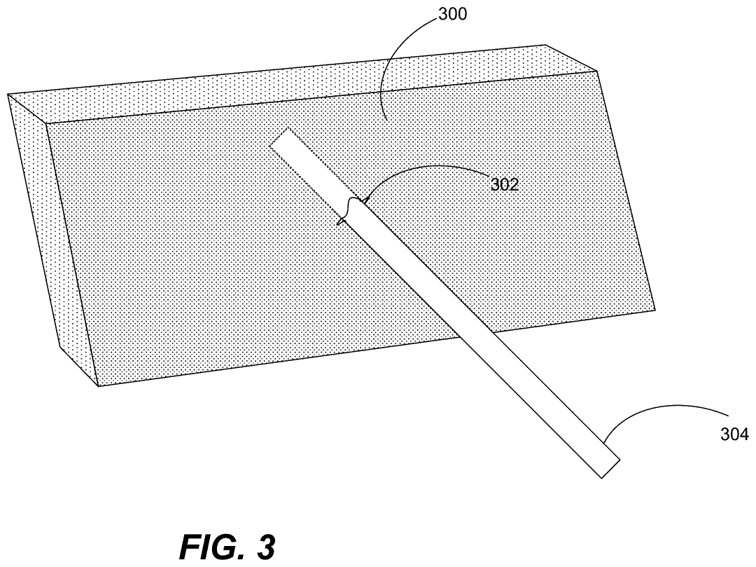

FIG. 3 is an illustration of an entry location 300 and deformable entry point 302 formed from foam, in accordance with some examples of the present disclosure. The foam may be of various substances that have pores or voids either occurring naturally, such as sea sponges, or produced from techniques such as introducing air bubbles into the material during the manufacture of the material. The foam may have pores of various sizes, including microscopic sizes. The foam may be designed to allow the insertion of a medical device 304 through a specific entry point, such as the deformable entry point 302, or other areas of the entry location 300. It should be noted that the material described in FIG. 3 and other figures described herein are merely exemplary and should not be considered as limiting the scope of materials that may be used.

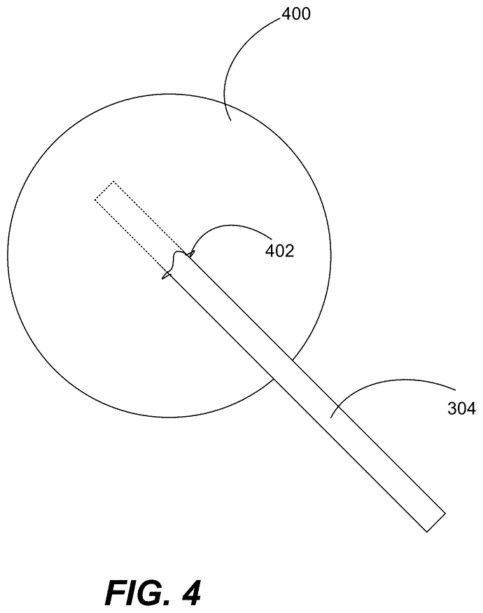

FIG. 4 is an illustration of an entry location 400 and deformable entry point 402 formed from silicone, or various forms of polymerized polysiloxanes or other polymers such as polypropylene, in accordance with some examples of the present disclosure. The silicones may be of various stiffness depending on the particular application. The presently disclosed subject matter is not limited to any particular type of silicone or to the use of silicone. Other materials that may be used in place of silicone may include various forms of rubber such as natural rubber, polyurethane, silicone rubber, neoprene, and others. In other examples, the material may be a composite made of an elastomeric film covered with a textile material on one side (such as a nonwoven, woven, or knitted fabric), illustrated in further detail in FIG. 8, below. The textile material, as well as other materials described herein, may have virucide properties. The presently disclosed subject matter is not limited to any particular type of rubber. Further, various other polymers may be used. Returning to the example provided in FIG. 4, the silicone may be designed to allow the insertion of a medical device 404 through a specific entry point, such as the deformable entry point 402, or other areas of the entry location 400.

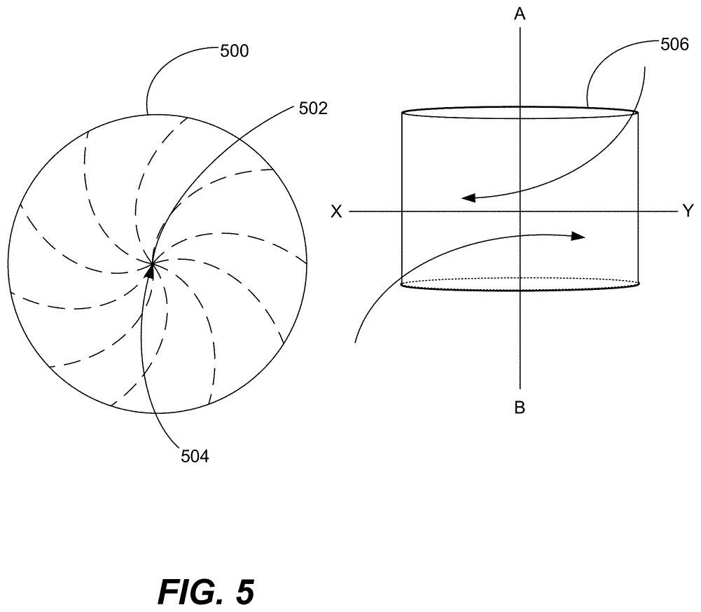

FIG. 5 is an illustration of an entry location 500 and deformable entry point 502 formed from a single material, in accordance with some examples of the present disclosure. The entry location 500 and deformable entry point 502 is comprised of an iris 504. The iris 504 is formed by twisting a cylinder 506 of flexible material (such as various plastics) around a central axis AB about a midline XY to form the iris 504. Medical devices may be inserted into and removed from the iris 504, as the iris 504 is opened from the outward pressure imparted on the deformable entry point 502 by the medical device and is closed when the medical device is removed. The characteristics of the barrier provided by the iris 504 may be varied depending on the deformability of the materials used as well as other factors such as the amount of twisting used to create the iris 504.

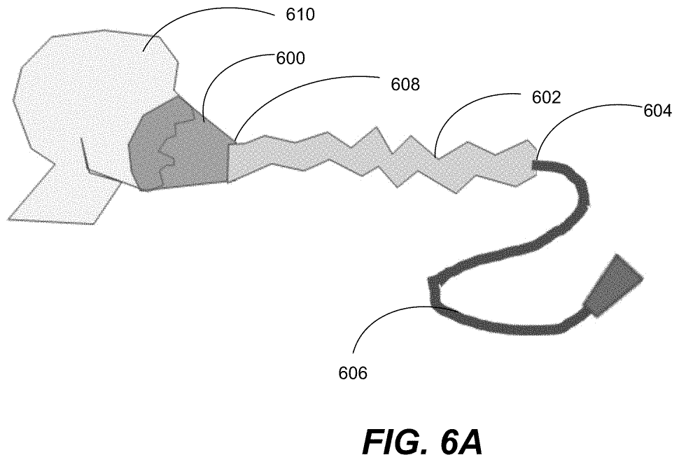

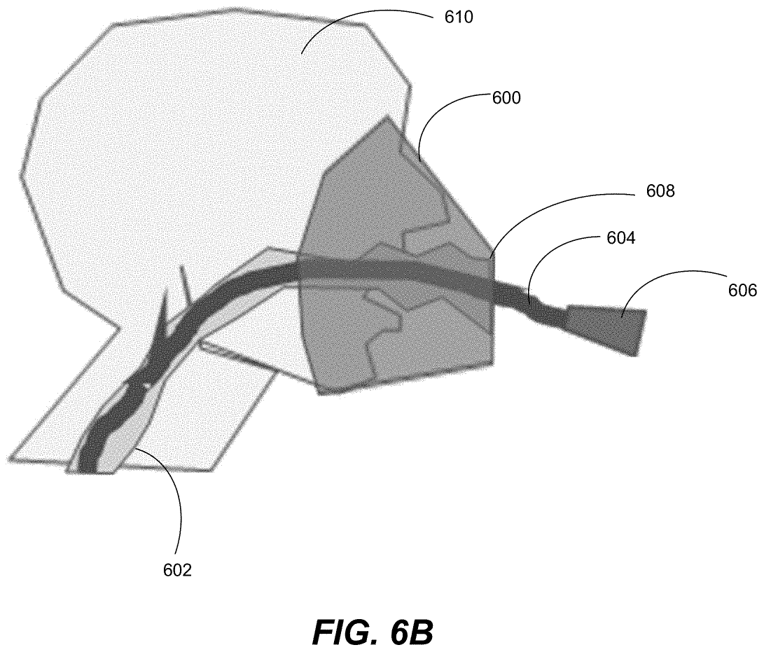

FIGS. 6A and 6B are illustrations showing a respiratory face mask 600 that may be used for procedures such as, but not limited to, endoscopy, in accordance with some examples of the present disclosure. As illustrated in FIG. 6A, the respiratory face mask 600 is attached to a sheath 602, an endoscopic fixation 604, and an endoscope 606 that runs through the interior volume of the sheath 602. As with other examples described herein, the mask includes an entry location 608, whereas the sheath 602 acts as a deformable entry point. The endoscope 606 is attached to the inside of the sheath 602 through the endoscopic fixation 604. It should be noted that the use of an endoscope is merely exemplary, as other types of medical devices may be used in a similar manner and are considered to be within the scope of the presently disclosed subject matter. As used herein, the term “insertable medical device” may be used to describe a general class of medical devices that are inserted into a patient's body, whereas an “endoscope” is a particular type of “insertable medical device.”

During use, the respiratory face mask 600 of FIG. 6A is designed to maintain a barrier between the environment outside of the respiratory face mask 600 and the environment inside the respiratory face mask 600. In this manner, the respiratory face mask 600 is designed to not only protect the patient 610 from germs introduced by the environment outside of the respiratory face mask 600, but also protect individuals from germs that are interior to the respiratory face mask 600 introduced by the patient 610 themselves.

FIG. 6B illustrates the respiratory face mask 600 after being inserted into the patient 610. As illustrated in FIG. 6B, the sheath 602 is inserted into the throat of the patient 610. Because the endoscope 606 has the endoscopic fixation 604, when the endoscope 606 is inserted into the throat, the sheath 602 turns inside out, whereby the surface of the sheath 602 that was outside when the endoscope 606 was not inserted is now the inner surface of the sheath 602 (turned inside-out). Thus, the barrier is maintained between the environment outside of the respiratory face mask 600 and the environment inside the respiratory face mask 600. When the endoscope 606 is extracted, the sheath 602 turns back outside-in in the manner illustrated in FIG. 6A. It should be noted that although FIG. 6B shows the insertion of the endoscope 606 into a throat of the patient 610, the endoscope 606 may also be inserted into a nostril of the patient 610.

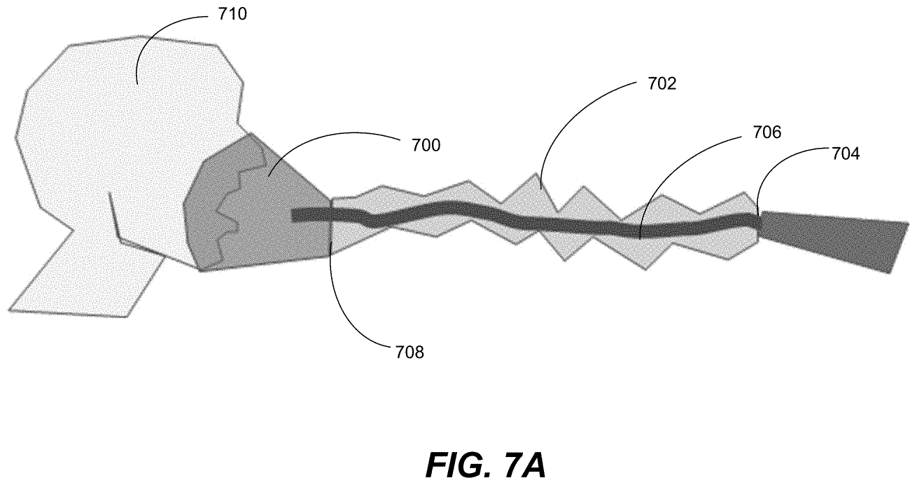

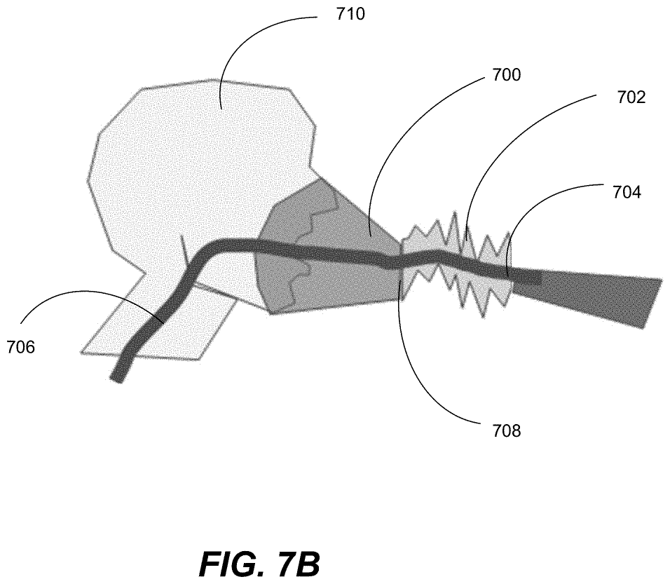

FIGS. 7A and 7B are illustrations showing an alternate design for a respiratory face mask 700 that may be used for procedures such as, but not limited to, endoscopy, in accordance with some examples of the present disclosure. It should be noted that endoscopy is used merely for purposes of illustrating an example use, as other medical devices and procedures may be performed in conjunction with various examples of respiratory face masks described herein and are considered to be within the scope of the presently disclosed subject matter. As illustrated in FIG. 7A, the respiratory face mask 700 is attached to a sheath 702, an endoscopic fixation 704, and an endoscope 706 that runs through the interior volume of the sheath 702. As with other examples described herein, the mask includes an entry location 708, whereas the sheath 702 acts as a deformable entry point. The endoscope 706 is attached to the inside of the sheath 702 through the endoscopic fixation 704. The endoscopic fixation 704 is placed closer to the end of the endoscope 706 controlled by a medical practitioner than the endoscopic fixation 604 of FIGS. 6A and 6B. In this manner, when inserted into the patient 710, the sheath 702 will not be pulled into the patient 710, but rather, will collect and compress outside of the patient, as illustrated in FIG. 7B.

FIG. 7B illustrates the respiratory face mask 700 after being inserted into the patient 710. In a manner different than FIG. 6B, while the endoscope 706 is inserted into the patient 710, the sheath 702 bunches between the mask 700 and the end of the endoscope 706 outside of the patient. However, in a manner similar to FIG. 6B, the barrier is maintained between the environment outside of the respiratory face mask 700 and the environment inside the respiratory face mask 700 during insertion and removal, providing a degree of protection for the patient 710 from the environment and medical personnel, but also the medical personnel from the patient 710. When the endoscope 706 is extracted, the sheath 702 extends back into the configuration illustrated in FIG. 7A. It should be noted that although FIG. 7B shows the insertion of the endoscope 706 into a throat of the patient 710, the endoscope 706 may also be inserted into a nostril of the patient 710.

FIG. 8 is a cross-sectional view of a sealing material 800 that may be used for a removable cover, such as the removable cover 122 of FIG. 1, in accordance with some examples of the present disclosure. The sealing material 800 can be removably affixed over a deformable entry point to reduce the probability of the introduction of particulate, germs, viruses, bacteria, and other matter into and around the deformable entry point before and after the penetration of a device into the deformable entry point. The example illustrated in FIG. 8 is a two-ply sealing material 800, where two layers form the sealing material 800. It should be understood, however, that other sealing materials 800 may be single-ply or more than two-ply materials.

The sealing material 800 of FIG. 8 includes an external textile layer 802 and an internal elastomeric film 804. The external textile layer 802 faces out from the direction of the person wearing the respiratory face mask, and, the internal elastomeric film 804 faces towards the face of the person wearing the face mask when removably affixed to a deformable entry point. The external textile layer 802 may be a knitted, woven or non-woven textile. In some examples, the external textile layer 802 may be impregnated or coated with biocidal, antimicrobial, and/or viricidal materials, or other materials as desired. The one or more materials impregnated or coated onto the external textile layer 802 may include, but are not limited to, fungicides, herbicides, insecticides, algicides, molluscicides, miticides, rodenticides, slimicides, germicides, antibiotics, antibacterials, antivirals, antifungals, antiprotozoals, and antiparasites. In some examples, the internal elastomeric film 804, or any internal layer used in alternate examples, may be coated or impregnated with similar materials.

The presently disclosed examples are considered in all respects to be illustrative and not restrictive. The scope of the disclosure is indicated by the appended claims, rather than the foregoing description, and all changes that come within the meaning and range of equivalents thereof are intended to be embraced therein.

What is claimed is:

1. A personal protective equipment, comprising:
a respiratory face mask having at least one void;
an entry location affixed to the at least one void of the respiratory face mask; and
a deformable entry point on the respiratory face mask, wherein the entry location and the deformable entry point are configured to allow an object to be inserted into and removed from the respiratory face mask to provide medical care.

2. The personal protective equipment of claim 1, wherein the respiratory face mask comprises an N95 respirator, a KN95 respirator, a bandana-type face mask, a pull-up face mask, a gaiter mask, a cloth mask, European standard FFP2 Mask, and a surgical mask.

3. The personal protective equipment of claim 1, wherein the entry location or the deformable entry point are formed from a foam.

4. The personal protective equipment of claim 1, wherein the entry location or the deformable entry point are formed from at least one type of polymerized polysiloxane or polypropylene.

5. The personal protective equipment of claim 1, wherein the entry location and the deformable entry point comprise an iris.

6. The personal protective equipment of claim 1, further comprising a gel coating on the entry location and the deformable entry point that is configured to maintain at least a portion of a seal when an object is inserted through the deformable entry point.

7. The personal protective equipment of claim 1, further comprising an anti-bacterial agent, an anti-microbial agent, or an anti-viral agent coated on the entry location or the deformable entry point.

8. The personal protective equipment of claim 1, further comprising a removable cover, the removable cover being removably affixable over the deformable entry point.

9. The personal protective equipment of claim 8, wherein the removable cover is comprised of an external layer and an internal layer.

10. The personal protective equipment of claim 9, wherein at least one of the external layer or the internal layer is coated or impregnated with one or more of a biocidal, antimicrobial, or viricidal material.

11. An endoscopic personal protective equipment, comprising:
a respiratory face mask;
a sheath affixed to the respiratory face mask, wherein the respiratory face mask and the sheath form an interior volume; and
an endoscope affixed to an endoscopic fixation location of the sheath, wherein the endoscope is present in the interior volume, wherein the sheath is moved into a throat or a nostril of a patient upon insertion of the endoscope into the throat or the nostril of the patient.

12. A personal protective equipment, comprising:
a respiratory face mask;
a sheath affixed to the respiratory face mask, wherein the respiratory face mask and the sheath form an interior volume; and
an insertable medical device affixed to a insertable medical device fixation location of the sheath, wherein the insertable medical device is present in the interior volume, wherein the sheath compresses between the insertable medical device fixation and the respiratory face mask upon insertion of the insertable medical device into a throat or a nostril of a patient.
